(19) United States
(12) Patent Application Publication
(10) Pub. No.: US 2010/0268177 A1
(43) Pub. Date: Oct. 21, 2010

(54) WOUND FILLERS

(75) Inventors: Kristian Hall, Hull (GB); Edward Hartwell, York (GB)

Correspondence Address: KNOBBE MARTENS OLSON & BEAR LLP, 2040 MAIN STREET, FOURTEENTH FLOOR, IRVINE, CA, 92614, US

(73) Assignee: Smith & Nephew Plc, London (GB)

(21) Appl. No.: 12/746,751

(22) PCT Filed: Nov. 17, 2008
(86) PCT No.: PCT/GB08/51070

§ 371 Date: Jun. 7, 2010

(30) Foreign Application Priority Data
Dec. 8, 2007 (GB) 0724039.3

(51) Int. Cl. A61M 1/00

(52) U.S. Cl. 604/313; 604/319

(57) ABSTRACT

A wound filling device is described and comprises a non-porous bag member, the bag member being sealed against ingress of wound exudate and being filled with resilient, compressible material.

The present invention relates particularly, though not exclusively, to non-porous wound filling members for use in topical negative pressure (TNP) therapy.

In recent years TNP therapy has become increasingly important in the field of improved treatment of wounds by making the healing thereof faster and more controlled.

The basic principle of TNP therapy is to create a closed cavity over the wound itself by means of a thin, flexible sealing film adhered to the patient's sound skin surrounding the wound; admitting one end of an aspirant conduit into the closed cavity, the conduit being sealed to the flexible film, for example; and connecting a distal end of the aspirant conduit to a vacuum source such as an electrically driven vacuum pump, for example, to create a pressure lower than the surrounding ambient atmospheric pressure within the wound cavity. As is known to the skilled person the lower pressure creates many beneficial therapeutic effects on the wound including increased blood flow to the wound and faster granulation of tissue, for example. There are very many variations on this basic principle of TNP therapy.

The types of wounds treated by TNP therapy generally range from quite small at about 5 cm² to very large traumatic wounds and burns of no particular maximum dimension. Such wounds may also have significant depth and therefore, significant volume. It is necessary to control the way in which a wound heals. For example, the wound should heal from the base up and close in from the edges desirably in a uniform manner. In particular it is desirable that the wound does not close over and form an occluded cavity in the flesh which is extremely undesirable from the patient's point of view as such form sites for infection.

To prevent the formation of occluded cavities during TNP therapy, the wound is usually packed with a filler which desirably has some resilience or “bounce” to resist the compressive forces created during TNP therapy by outside ambient atmospheric pressure bearing down on the wound due to the vacuum created in the wound cavity. The purpose of the filler is to keep the surrounding edges of the wound apart so that they cannot grow over and form a cavity. The filler may also perform the function of providing fluid flow channels between the wound and the filler in order to provide a uniform reduced pressure distribution over the surface area of the wound and to promote efficient aspiration of exudate fluids away from the wound surface and generally into a remote waste receptacle associated with the aspirant conduit.

Typically, many TNP systems sold today use either foam or gauze as the wound filling medium and which has the disadvantage of tissue ingrowth into the pores of the filler and which may cause damage to newly formed granulation tissue on removal and also pain to the patient. Furthermore, such porous materials are subject to becoming saturated with wound exudate fluids causing a build up of bacteria leading to infection.

It is an object of the present invention to at least reduce or minimise the disadvantages of known wound filling materials.

According to the present invention there is provided a wound filling device comprising a non-porous bag member, the bag member being sealed against ingress of wound exudate and being filled with resilient, compressible material.

As noted above the basic principle of TNP therapy is to create a closed cavity over the wound itself by means of a thin, flexible sealing film adhered to the patient's sound skin surrounding the wound; admitting one end of an aspirant conduit into the closed cavity, the conduit being sealed to the flexible film, for example; and connecting a distal end of the aspirant conduit to a vacuum source such as an electrically driven vacuum pump, for example, to create a pressure lower than the surrounding ambient atmospheric pressure within the wound cavity.

Desirably, the bag member may be provided with a surface texture such as may permit the provision of a uniform vacuum pressure distribution, when used in TNP therapy, over the surface of a wound bed and also allow wound exudate to be drawn from all parts of the wound bed towards an aspiration conduit so as to be removed from the site of a wound.

Suitable surface textures may be provided by, for example, but not limited to pimples, channels and ribs and the like formed on the bag member surface. The gaps and channels formed between protrusions of various types on the bag surface permit a uniform pressure distribution over the area of the wound bed and also provide fluid pathways to permit wound exudate to be drawn around the bag member rather than through it as with prior art wound fillers and be aspirated away from the wound site.

The textured surface also applies micro-stresses to the wound bed surface and which has been shown to stimulate tissue growth and wound healing.

Advantageously it has been found that fluid channels and pathways on the surface of the bag member may be of the order of about 0.5 to 2.0 mm with struts (i.e. ridge members, for example, between channels) of the order of 0.1 to 1.0 mm. A non-limiting example may be upstanding cylinders on the bag member surface of 0.2 mm diameter and 1 mm height and pitched centres of about 1 mm arranged in equilateral triangular repeated array.

Materials from which the bag member may be formed is desirably soft and conformable may suitably be selected from the group including: HDPE, PU such as Elastane SP806 (trade mark), silicone, PVC and EVA, for example. The bag member may also be of laminated construction such as from, for example, Biobrane (trade mark) which comprises a layered structure of non-porous silicone rubber; a Nylon (trade mark) mesh adhered thereto; and collagen peptide particles impregnated into the Nylon fabric mesh surface which is placed adjacent the wound.

The bag member wound packing member may also advantageously be treated with biologically active components such as silver or in addition to or in substitution of silver, antimicrobial compounds such as e.g. silver containing species, iodine, guanidines, biguanidines etc., analgesics e.g. aspirin, ibuprofen etc., anaesthetics e.g. amethocaine, lignocaine etc., growth factors e.g. VEGF PDGF, vasodilators e.g. NO, sildenafil, histamine etc. for example. Other examples of suitable active components may be found in WO 2005/105180 the content of which is included herein by reference.

The bag member of the wound filling devices according to the present invention may optionally be soaked or coated in a non-adherent gel such as, for example, a petroleum gel an example of which is Cuticerin (trade mark), silicone gel or a hydrogel.

The resilient, compressible filling of the bag member may comprise foam or polystyrene beads, for example, however any suitable material having the required physical characteristics may be used as the bag filling material does not come into contact with wound exudate. The bag filing material should be relatively soft but hard enough to deflect skin/tissue in the wound bed so as to stress the tissue without being too aggressive. Suitably a material hardness in the region of 70 to 100 Shore may be employed. It has been found that a plain smooth bag even with the filling may not be able to resist being pressed down into intimate contact with the wound bed by the surrounding atmospheric pressure and occluding areas of the wound from the vacuum in the wound cavity.

In some embodiments of the present invention the resilient, compressible filling in the bag member may be a gas such as air or nitrogen, for example, in order to save weight on a patient in the case of a large wound. In such cases it may be desirable for the surface texturing of the bag member to be more pronounced.

The wound filling device(s) of the present invention may be made to work or stress a healing wound by cycling of the vacuum applied to the wound cavity resulting in the surrounding atmospheric pressure stressing the wound bed in a cyclic manner.

A plurality of wound filling devices according to the present invention may be joined together in a string and a suitable number to fill a given wound be cut off from the string. Suitable material with which to string a plurality of wound filling devices together may be nylon filament which does not shed debris in the wound. A particular advantage of the wound filling devices of the present invention is that they are used as units and not sub-divided and do not like foams and gauzes shed debris into the wound when cut and which debris can be occluded in a healing wound and cause later infection.

Although the bag member is sealed against ingress of wound exudate, in an embodiment of a wound filling device according to the present invention, the interior of the bag member may be connected by a flexible conduit to a fluid reservoir outside the wound and the covering sealing drape. The reservoir may contain a heated fluid such as warm water, for example, which may be beneficial in terms of wound healing. Alternatively, the reservoir may contain a cooled fluid which in some circumstances may be more beneficial to the wound healing process. Some circulation of the fluid may be achieved by pressure cycling of the vacuum in the wound cavity which may cause fluid to be drawn into the bag member in one cycle and be expelled in the following cycle. Thus, if the fluid in the reservoir is temperature controlled so may the fluid in the bag member in the wound.

In order that the present invention may be more fully understood, examples will now be described by way of illustration only with reference to the accompanying drawings, of which:

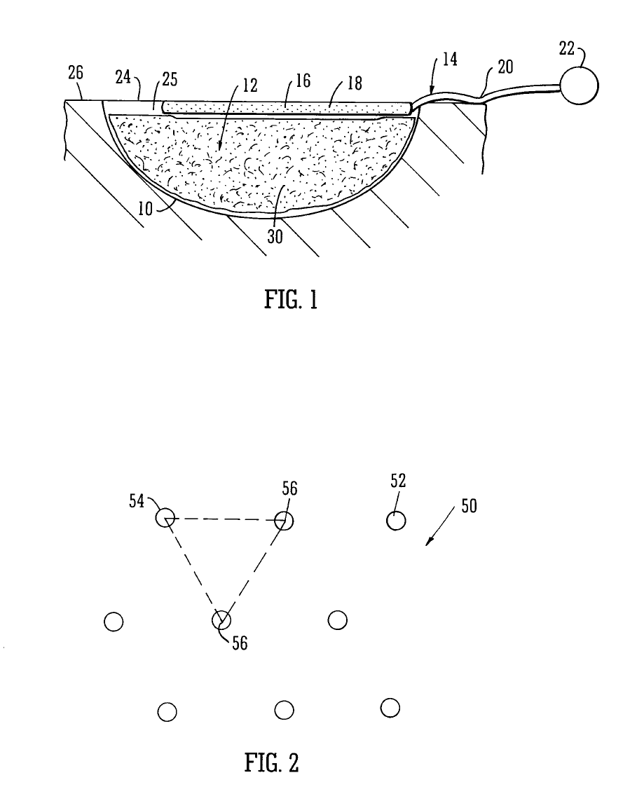

FIG. 1 shows a schematic cross section of wound having a wound filling device according to the present invention therein as part of a TNP therapy apparatus;

FIG. 2 shows a schematic of a suitable bag member surface;

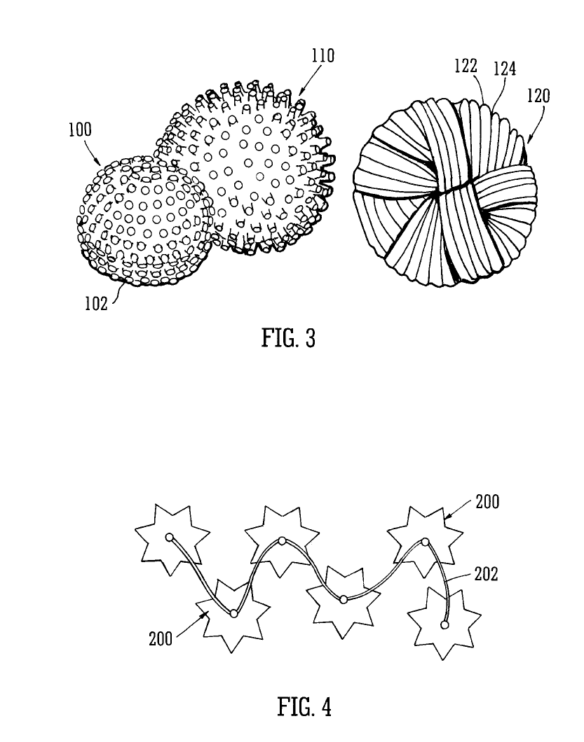

FIG. 3 shows a photograph of examples of wound filling devices;

FIG. 4 shows a schematic of a plurality of wound filling devices strung together; and

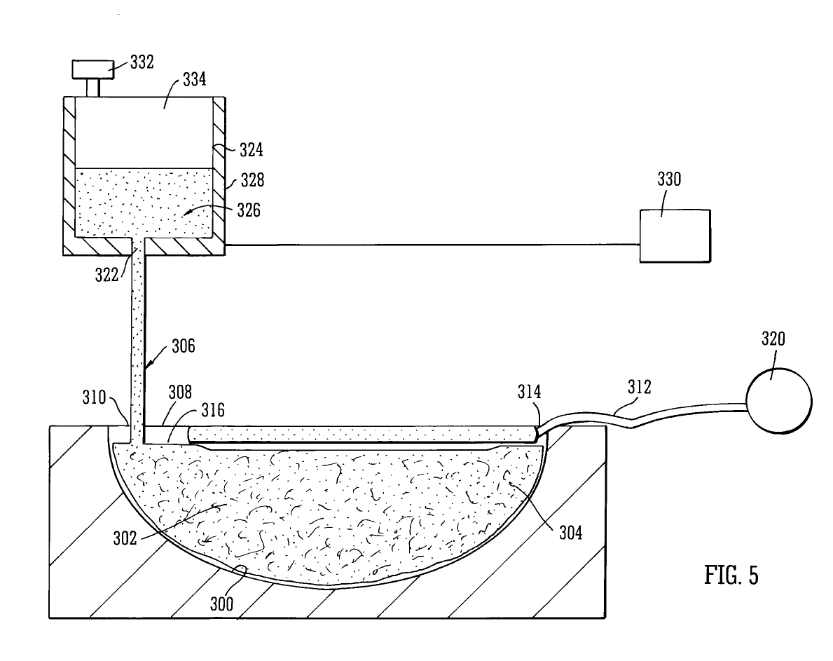

FIG. 5 which shows a schematic cross section of a wound having an alternative embodiment of a wound filing device to that shown in FIG. 1.

Referring now to the drawings and where the same features are denoted by common reference numerals.

FIG. 1 shows a schematic representation of a wound 10 having a wound filling device 12 according to the present invention therein. The wound filling device 12 has a drain conduit 14 lying on the top thereof, the drain conduit having a portion 16 having a plurality of apertures 18 to aspirate wound exudate within the wound 10 into a second, single lumen portion 20 of the drain conduit which is operably connected to a vacuum pump 22. A sealing drape 24 overlies the wound 10 to create a sealed wound cavity 25, the sealing drape having a layer of pressure sensitive adhesive (not shown) in known manner on the side which contacts and adheres to sound flesh 26 surrounding the wound 10. The apparatus may also contain in known manner a waste canister (not shown) to receive wound exudate and a power source and control system (both not shown) for the vacuum pump 22.

In this embodiment, the wound filling device 12 comprises a sealed bag member 30 being filed with polystyrene beads of a size range 0.1 to 20 mm preferably 1.0 to 10.0 mm. The bag material is embossed (not shown) EVA sheet, cut to size and welded at a seam (not shown) after filling with the beads. In operation and since the bag member 30 is not porous or otherwise permeable, wound exudate is drawn around the outer surface of the bag member towards the drains conduit portion 16 from whence it is aspirated away from the wound site through the conduit 20 in known manner. Due to the vacuum produced by the pump 22 and the surrounding ambient atmospheric pressure, the wound filling device 12 is pressed into intimate contact with the wound surface, however, an even vacuum depression is achieved over the whole wound surface due to the embossed surface texture of the EVA material and the contained polystyrene beads providing a multitude of channels along which exudate may be aspirated. Thus, the interior of the wound filling device 12 and the contained beads do not become filled or saturated with wound exudate and which lessens the bacterial load at the wound 10 thus also lessening the risk of infection.

FIG. 2 shows a schematic of a pattern of surface features suitable to form the surface texture of bag member of a wound filling device according to the present invention. The pattern denoted generally at 50 possesses a multitude of upstanding silicone rubber cylinders 52 (shown in plan view only) of height about 1 mm and diameter 54 of 0.2 mm and having pitched centres 56 1 mm apart and formed in an equilateral triangular array. The silicone rubber material is soft and the cylinders 52 cause no discomfort or trauma to the patient but do provide a multitude of paths for fluid flow.

FIG. 3 shows a photograph of three examples of wound filling devices according to the present invention. Device 100 comprises a hollow gas filled moulded ball of polyurethane material having a surface similar to that described with respect to FIG. 2 but with cylindrical protrusions 102 having lower aspect ratio. Device 110 comprises blow moulded polyurethane (Elastane SP806—trade mark). Device 120 comprises a moulded polyurethane material ball having a surface provided with ridges 122 and channels 124 in varying orientations.

The wound filling devices in FIG. 3 may also have passages or holes therethrough with the proviso that such passages or holes do not allow exudate to communicate with interior volumes of the ball which may contain gas or beads, for example, of resilient, compressible material.

FIG. 4 shows a plurality of schematic wound filling devices 200 strung together by a thread 202. Such a string of devices may be sold as a kit having a plurality of, say twenty, devices thereon and the clinician merely cuts off the number which is appropriate to filling the wound.

FIG. 5 shows a schematic alternative embodiment of the wound filling device shown in FIG. 1. A wound 300 has a wound filling device 302 therein of a size to fill the wound. The construction of the device 302 is essentially the same as that of the device shown and described with reference to FIG. 1 save for the fact that the bag member 304 has a flexible conduit 306 attached thereto to communicate with the interior (not shown) of the bag member 304 and which conduit passes sealingly through the sealing drape 308 at the point 310 thus, no wound exudate is able to enter either the conduit 306 or the interior of the bag member 304. An aspirant conduit 312 passes sealingly through the drape 308 at point 314 thus creating a sealed wound cavity 316 beneath the drape 308 apart from the connection of the conduit 312 to a vacuum pump 320 and any other apparatus features such as a waste canister and associated equipment which may be present in known manner. The conduit 306 is connected at its distal end 322 to a reservoir 324 which contains warm water 326 which is maintained at a constant temperature by a heating jacket 328 which has an associated power supply and control system 330. The reservoir 324 is essentially closed to ambient atmosphere but has a 0.2 micron filter 332 to prevent bacteria from entering an air space 334 but which permits pressure in the air space 334 above the water to be maintained at ambient atmospheric pressure. The warm water 326 fills the bag member 304 and has valuable therapeutic effects on the wound 300. The water 326 in the bag member 304 may be circulated to an extent by pressure pulsing of the wound cavity 316. As the vacuum in the wound cavity is increased (i.e. lower pressure relative to ambient) the surrounding ambient atmospheric squeezes the bag member 304 to expel some of the water back to the reservoir 324; relaxation of the vacuum allows the bag member to expand thus drawing water at the controlled temperature into the bag member.

Alternatively, in some circumstances it may be beneficial for the fluid 326 to be cooled rather than heated and the apparatus described above contemplates cooling rather than heating of the fluid 326.

The bag member 304 may be provided with a stub conduit sealed by a clamp and a separate conduit appropriate to the associated apparatus layout described above attached to the stub by known means.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A wound filling device comprising a non-porous bag member, the bag member being sealed against ingress of wound exudate and being filled with resilient, compressible material characterised in that an interior of the bag member is provided with means to connect a fluid supply thereto.

2. A wound filling device according to claim 1 wherein the bag member is provided with a surface texture.

3. A wound filling device according to claim 2 wherein the surface texture permits a uniform pressure distribution to be achieved, in use, in a wound.

4. A wound filling device according to claim 2 wherein the surface texture permits uniform fluid flow over an area of a wound.

5. A wound filling device according to claims 2 wherein the surface texture is selected from the group consisting of cylindrical-shaped protrusions, pimples, channels, pathways and ribs.

6. A wound filling device according to claim 5 wherein fluid channels and pathways on the surface of the bag member are about 0.5 to 2.0 mm deep.

7. A wound filling device according to claim 1 wherein the bag member is made from a material selected from the group consisting of HDPE, PU, silicone, and EVA.

8. A wound filling device according to claim 1 wherein the resilient, compressible filling of the bag member comprises foam or polystyrene beads.

9. A wound filling device according to claim 1 wherein the filing of the bag member has a material hardness in the region of 70 Shore.

10. A wound filling device according to claim 1 wherein the fluid supplied to the interior of the bag member is a heated fluid.

11. A wound filling device according to claim 1 wherein the fluid supplied to the interior of the bag member is a cooled fluid.

12. A wound filling device according to claim 10 wherein the supplied fluid is a liquid.

13. A wound filling device according to claim 1 wherein the resilient, compressible filling is a gas.

14. A wound filling device according to claim 1 wherein the outer surface of the bag member is treated with a biologically active component.

15. A wound filling device according to claim 1 wherein the bag member is treated with a chemical non-adherent to tissue.

16. A wound filling device according to claim 1, wherein the wound filling device comprises at least one addition wound filling device attached thereto.

17. A wound filling device according to claim 16 wherein the wound filling device is attached to the at least one additional wound filling device with a thread.

18. A wound filling device according to claim 17 wherein the thread is a polymer monofilament.

19. (canceled)

20. (canceled)

21. (canceled)
